(19) United States
(12) Patent Application Publication
(10) Pub. No.: US 2014/0194826 A1
(43) Pub. Date: Jul. 10, 2014

(54) DRUG DELIVERY INJECTION PEN WITH ADD-ON DOSE CAPTURING AND DISPLAY MODULE

(71) Applicants: Ole Christian Nielsen, Hilleroed (DK); Morten Soerensen, Ballerup (DK)

(72) Inventors: Ole Christian Nielsen, Hilleroed (DK); Morten Soerensen, Ballerup (DK)

(73) Assignee: NOVO NORDISK A/S, Bagsvaerd (DK)

(21) Appl. No.: 14/127,723

(22) PCT Filed: Jul. 9, 2012
(86) PCT No.: PCT/EP2012/063349

§ 371 Date: Feb. 20, 2014

Related U.S. Application Data
(60) Provisional application No. 61/507,223, filed on Jul. 13, 2011.

(30) Foreign Application Priority Data
Jul. 7, 2011 (EP) 11173082.6

(51) Int. Cl. A61M 5/315

(52) U.S. Cl. CPC A61M 5/31553; A61M 5/31568
USPC 604/189; 604/208

(57) ABSTRACT

A drug injector (1) comprising expelling means for expelling a dose of drug from a reservoir, a release member (13) for releasing the drug expelling means, and an actuation member (33) adapted to be moved by a user between an initial, intermediate and actuated position in which the release member is moved to release the drug expelling means. The injector further comprises an electronic capturing system for capturing data representing a dose of drug to be expelled, and a switch (38) for starting initialization of the data capture system, the switch being actuated when the actuation member is positioned in its intermediate position. A spring (34) provides a biasing force against movement of the actuation member between its initial and intermediate position. Thereby the electronic data capturing system is allowed to initialize during the actuation member's movement between the intermediate and the actuated position.

The present invention relates to a system for capturing drug delivery data. Especially, the invention addresses the issue of generating a triggering signal for an electronic data capturing system.

### BACKGROUND OF THE INVENTION

In the disclosure of the present invention reference is mostly made to the treatment of diabetes by delivery of insulin, however, this is only an exemplary use of the present invention.

Drug Injection devices have greatly improved the lives of patients who must self-administer drugs and biological agents. Drug Injection devices may take many forms, including simple disposable devices that are little more than an ampoule with an injection means or they may be durable devices adapted to be used with pre-filled cartridges. Regardless of their form and type, they have proven to be great aids in assisting patients to self-administer injectable drugs and biological agents. They also greatly assist care givers in administering injectable medicines to those incapable of performing self-injections.

Performing the necessary insulin injection at the right time and in the right size is essential for managing diabetes, i.e. compliance with the specified insulin regimen is important. In order to make it possible for medical personnel to determine the effectiveness of a prescribed dosage pattern, diabetes patients are encouraged to keep a log of the size and time of each injection. However, such logs are normally kept in handwritten notebooks, from the logged information may not be easily uploaded to a computer for data processing. Furthermore, as only events, which are noted by the patient, are logged, the note book system requires that the patient remembers to log each injection, if the logged information is to have any value in the treatment of the patient's disease. A missing or erroneous record in the log results in a misleading picture of the injection history and thus a misleading basis for the medical personnel's decision making with respect to future medication. Accordingly, it may be desirable to automate the logging of ejection information from medication delivery systems.

Though some injection devices integrate this monitoring/acquisition mechanism into the device itself most devices of today are without it. The most widely used devices are purely mechanical devices either durable or prefilled. The latter devices are to be discarded after being emptied and so inexpensive that it is not cost-effective to build-in electronic data acquisition functionality in the device it-self. Correspondingly, data acquisition/monitoring functionality have been proposed to be provided in a separate device to be put on or in the injection device, i.e. some kind of accessory e.g. an add-on module to the injection device.

For example, WO 2010/098927 discloses a medical module which is configured to be attached to a drug delivery pen, the module being adapted to detect and store selected and ejected dosages as well as other data.

Having regard to the above, it is an object of the present invention to provide systems and methods supporting cost-effective as well as energy-effective detection and storage of data related to use of a drug delivery device.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect of the invention a drug delivery system is provided, comprising expelling means for expelling a dose of drug from a reservoir, an electronically controlled capturing system for capturing data representing a property related to the amount of drug expelled from a reservoir by the expelling means, and switch means for starting initialization of the capturing system. The system further comprises a release member for releasing or driving the expelling means and adapted to be moved between (a) an initial position, and (b) an actuated position in which the expelling means is released or driven, and an actuation member adapted to be moved by a user between (i) an initial position, (ii) an intermediate position in which the switch means is actuated to start initialization of the capturing system, and (iii) an actuated position in which the release member is actuated, as well as actuation biasing means providing a biasing force against movement of the actuation member between its initial and intermediate position. By this arrangement the capturing system is allowed to initialize during the actuation member's movement between the intermediate position and the actuated position—indeed, the intermediate and actuated positions are not identical. The biasing means assures that a positive distance of travel is provided between the initial and intermediate positions, just as the biasing means allows the system to provide a resistance to travel of the actuation member which helps assure that a user will not force down the actuation member too swiftly. When providing a data capture system, it should be secured that the system is operational in good time before the system shall make its data acquisition (of e.g. out-dosing amount and out-dosing time) in order to get the electronics ready for the measurement(s). Initialization may include power-up time for the electronics of a fully or partly dormant system, the time for initializing the electronics or the necessary time for the electronics to make a reliable measuring/data acquisition.

By the above arrangement an energy-effective system is provided which is energized just prior to out-dosing (i.e. when the user actuates the actuation member to its intermediate position), yet sufficiently early to allow the electronic data capture system to capture data prior to the expelling means being released or driven.

As appears, two actuatable members are provided, the release member actuating the expel- ling mechanism per se, the other being directly actuated by the user. In case the drug delivery system of the invention is provided with an integrated system, the first actuation member may be an internal part not visible to the user, however, in case the system is an add-on unit adapted to be used with a traditional drug delivery device having e.g. an end-mounted actuation button on a pen-formed device, the add-on unit may comprise an additional actuation button adapted to be arranged in a “button-on-button” relationship, this allowing the device to be used in the same way as hitherto. For such an assembly, a trigger mechanism interfacing to the push-button of the e.g. injection pen must not jeopardize the original and primary function of the pen and push-button.

The drug expelling means may be designed to be set to deliver a variable user selected dose size, the data capture system correspondingly being adapted to capture the set and/or expelled dose, however, the data capture system may also be used with a fixed dose drug delivery device or to capture time only, the data capture system thus primarily logging time as a function of detected out-dosing.

The drug expelling means (or mechanism) may be of the type in which a spring is loaded during setting of a dose, this allowing spring-driven drug expelling when the set and loaded mechanism is released by the user, this allowing a design in which the release (and actuation) button is axially stationary during dose setting. Alternatively, the drug delivery system may be of the traditional type in which a member is dialled proximally to set a dose and subsequently moved distally to expel the set dose. In such a system the expelling means can be said to be driven when the member starts to be moved distally.

In an exemplary embodiment the drug expelling means can be set to deliver a user selected dose size, the selected dose size being represented by indicia which can be captured by the detection means. For example, the drug expelling means may comprise a rotatable member on the surface of which indicia are arranged, the indicia representing the set dose size being positioned at a given capture position allowing capture thereof by the detection means. The rotatable member may be adapted to rotate during expelling of a set dose, the indicia at a given point in time representing, directly or indirectly, the expelled dose. Such a design corresponds to the traditional design of a pen-formed drug delivery device, the indicia being numbers, e.g. 0-80, being arranged on a rotatable drum, the actually set dose being represented by a digit, e.g. 20, showing in a window. When the indicia are numerical values which can be read by a user, also the capturing system can be designed to capture the information represented by the digits.

Alternatively, two indicia may be provided on one or two rotatable members, one indicia being readable by a user, the other being readable by the capturing system. In such a system the second set of indicia designed to be captured by the electronic means could be arranged on a part of the expelling mechanism normally not visible to the user.

Other means for communicating and capturing data related to dose size may be implemented. For example, the pen may be provided with electronic means generating a wireless signal to be captured by the capturing system, it may be provided with magnetic means or the pen may produce an audible signal to be captured by the capturing system.

For the above-described embodiments, the actuation member may, directly or indirectly, be brought in contact with the release member before or after it has reached the intermediate position, e.g. by a “button-on-button” design. The biasing means may comprise a spring acting on the actuation member, the spring having a stationary portion being supported by a component of the system which does not move during setting and expelling of a dose of drug.

For the above-described embodiments, the drug delivery system may be pre-filled comprising a drug reservoir not intended to be replaced after it has been emptied, or the system may be configured to receive a drug reservoir in the form of a replaceable drug cartridge, e.g. comprising a cartridge holder. In an exemplary embodiment the above-described features may be provided in a drug delivery system comprising a drug delivery unit and a data capture unit releasably attachable to each other, the drug delivery unit comprising the expelling means, and the release member, whereas the data capture unit comprises the actuation member, the electronically controlled data capture system, the switch means, and the biasing means. Alternatively, an integrated system comprising a housing in which the expelling means, the release member, the actuation member, the electronically controlled capturing system, the switch means, and the actuation biasing means are arranged.

In a further aspect a drug delivery system is provided, comprising a drug reservoir or means for receiving a drug reservoir, drug expelling means for expelling a dose of drug from the reservoir, a first actuation member for driving or releasing the drug expelling means, a second actuation member adapted to be moved by a user between (i) an initial position, and (ii) an actuated position in which the first actuation member is moved to drive or release the drug expelling means, and biasing means providing a biasing force against movement of the second actuation member between its initial and intermediate position, the biasing means thereby providing a means to take up slack and adapt to tolerances between the two actuation members. Corresponding to the first aspect, the second button may be provided as part of a data capture unit releasably attachable to a drug delivery unit. Depending on the design, initialization of the data capture unit may be different from the switch design described as part of the first aspect. The biasing means may be in the form of the same means as disclosed for the first aspect.

As used herein, the term “insulin” is meant to encompass any drug-containing flowable medicine capable of being passed through a delivery means such as a cannula or hollow needle in a controlled manner, such as a liquid, solution, gel or fine suspension, and which has a blood glucose controlling effect, e.g. human insulin and analogues thereof as well as non-insulins such as GLP-1 and analogues thereof. In the description of the exemplary embodiments reference will be made to the use of insulin.

### BRIEF DESCRIPTION OF THE DRAWINGS

In the following the invention will be further described with reference to the drawings, wherein

FIG. 1 shows a proximal portion of a pen-type drug delivery device,

FIG. 2 shows a first embodiment of a drug delivery device with an add-on switch arrangement,

FIG. 3 shows a second embodiment of a drug delivery device with an add-on switch arrangement,

FIG. 4 shows a third embodiment of a drug delivery device with an add-on switch arrangement,

FIGS. 5 and 6 show a pen-type drug delivery device in combination with an add-on dose logging module, and

FIG. 7 shows a schematic representation of a vision system for a curved dose drum.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. For example, the exact position of the switch in FIGS. 2, 3 and 4 is only shown schematically and does not necessarily indicate the most appropriate location of such a switch relative to an actuation member for any given design.

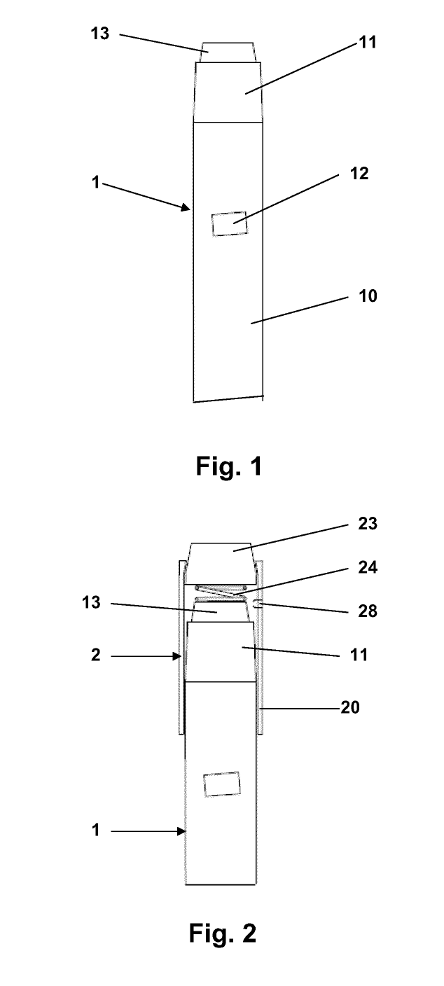

FIG. 1 shows the proximal portion (i.e. comprising a dose setting and mechanical expelling mechanism but not a drug reservoir or cartridge) of a traditional pen-formed drug delivery device as used typically for administration of insulin. The shown embodiment represents the type of pen in which a spring is loaded during setting of a dose, this allowing spring-driven drug expelling when the set and loaded mechanism is released by the user, this allowing a design in which the release (or actuation) button is axially stationary during dose setting. A more detailed description of such a pen can be found in e.g. US 2009/054839, US 2008/306446 and US 2008/234634 which are hereby incorporated by reference.

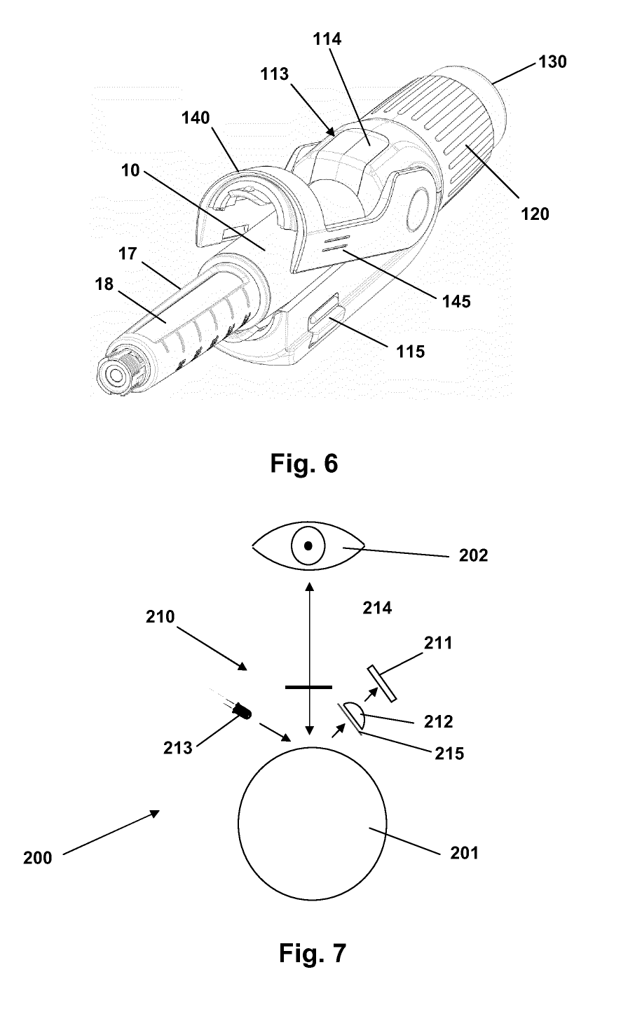

More specifically, FIG. 1 shows a drug delivery device 1 of the pen type comprising a proximal part having a mainly cylindrical housing portion 10 with an expelling mechanism and a distal portion (see FIG. 6) comprising a drug cartridge with an axially moveable piston driven by the expelling mechanism. The pen comprises a rotatable dose setting ring member 11 allowing a user to set and adjust (i.e. dial-up and dial-down) a variable dose size of given increments (e.g. 1 IU insulin) to be expelled from the cartridge, the actual dose size (e.g. 25 IU insulin) being indicated by numbers shown in a window 12, the numbers being arranged on a rotating dose drum member. The maximum amount of drug that can be delivered during one out-dosing is defined by the injection device. For example the injection device may deliver variable dose amounts during one out-dosing between 1 IU insulin and 80 IU insulin.

A release member in the form of push button 13 is arranged at the proximal end and adapted to release the expelling mechanism when pushed distally by the user. As the mechanism is released the set dose will be expelled from the cartridge and the dose drum will correspondingly rotate back to its initial zero position. If the mechanism is designed to stop expelling when the user stops pushing the release button, the number display in the window will show the portion of the dose (e.g. the numbers of units) not yet expelled, e.g. 10 units of insulin.

Based on the above pen design, an electronic data acquisition system could be based on the following concept: To save energy the system is dormant during non-use of the device as well as during setting of the dose. When the dose is thus set and the user actuates the release button this indicates to the system that the set dose is the actual dose to be captured. However, depending on the principle used to capture information about the set dose, the system may need time to “wake up” and capture dose information before the information “is gone”, i.e. the only information representing the set (and expelled) dose is the position of mechanical components, e.g. the dose drum. This would for example be the case in a system based on optical detection of information arranged on the dose drum, e.g. the numbers shown to the user or “hidden” information (see below). In case the pen is designed to output a signal during expelling (e.g. pulses corresponding to a certain dose amount), the capturing system may miss part of the information if it is not fully ready when expelling starts.

In a straight-forward application of such a system onto an existing pen device, the wake-up would have to take place during the time of travel for the release button between its initial position and the position in which the expelling mechanism is released, i.e. given such a travel is provided. In case the system is provided as an add-on unit to be mounted onto a standard pen, the add-on unit could be provided with a secondary actuation push-button adapted to directly engage the pen release push-button, i.e. “hard-mounted”. Apart from requiring close tolerances, such a design would rely on the same (short) button travel.

Addressing the above issue, FIG. 2 shows an embodiment in which a pen device of the type shown in FIG. 1 is provided with an add-on data capture module 2 (or unit) providing additional “wake-up time” when released. The add-on module comprises a housing 20 adapted to releasably engage the pen body in a pre-determined position, electronic detection means (not shown), a switch 28 (e.g. electro mechanical, inductive or optical), a secondary user-operated actuation member (in the form of push-button 23) and a biasing spring member 24.

As the actuation push-button is spaced axially relative to the primary release button the arrangement elongates the movement of the add-on module's actuation push button, this making the overall system less sensitive to tolerances as well as providing more time for the controller system to power-up or initialize or achieving reliable data measurements.

FIG. 2 shows a system with a biasing means in the form of a compression spring 24 (here a helical compression spring) arranged in serial between the two push-buttons 13, 23 (the one from the add-on module and the other from the injection device). In a situation of use, a user pushes the add-on module push button e.g. by a finger; hereby performing a mechanical work W equal the integrated multiple of force (F) and travel (d). For simplicity here explained with a constant force F; i.e. W=F*d.

For the user to perform the necessary work s/he must activate the add-on actuation push button with a force over a distance. The longer distance the more time it will take to push down the push button. And the longer distance the more insensitive is the system for tolerance mismatch between the injection device push button travel to initiate or enable the out-dosing and the add-on module push button travel. For example, the serial spring can be designed to allow the trigger push button to travel between 1.5 and 5 times the add-on module push button travel before the necessary force is build on the injection device's push button to release the expelling mechanism, or to travel between 5 and 15 times the travel of the injection device push-button or more. The longer travel of the add-on device's push button the more insensitive is the system for tolerances in the injection device's out-dosing trigger force level in order to get robust switch distances for the controller system. Together with the reduced sensitiveness due the longer travel it will also take more time for the user to trigger/activate the injection device by pushing down the add-on module's push button. In this serial spring system the total amount of force on the add-on actuation push-button to trigger the out-dosing in the injection device is the same as when there is no add-on module but the force instead is applied directly on the injection devices release button.

In the system shown in FIG. 2 there may still be some influence from the injection device mechanical tolerances and build-in spring mechanism (if the device is of the type disclosed in US 2009/054839 or US 2008/306446) on the applied force to trigger an out-dosing and thereby some influence on when the add-on module switches is activated relative to the depressing of the injection device push button by the series spring. When depressing the add-on module push button the add-on module series compression spring must first overcome the pretension of an internal spring in the injection device before the injection device push button moves and next be further compressed to build up the force to move the injection device push button to its out-dosing triggering/enabling position.

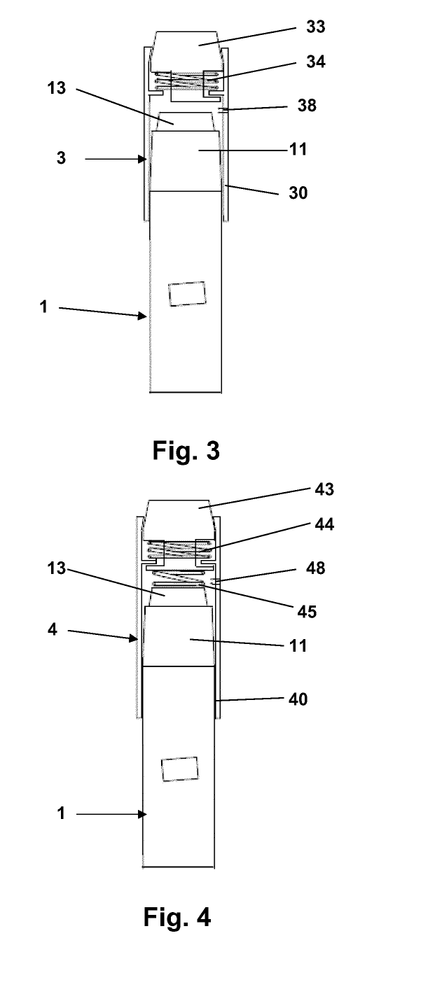

Correspondingly, FIG. 3 shows a system with a drug delivery device 1 as in FIG. 1 and an add-on data capture module 3 comprising a compression spring 34 working in parallel only on the add-on module's push button 33 and where the influence though normally small from the injection device's out-dosing force trigger level and mechanics is removed to only impact the system when the travel of the add-on module's push button reaches the injection device release push button 13 (directly or indirectly through another mechanical part). The spring is supported between the button 33 and an inner circumferential flange 35 of the data capture module. The benefit of this system compared with the one shown in FIG. 2 is the less susceptibility of the injection device tolerance on trigger force level on the travel of the add-on module's push button. A disadvantage relative to the embodiment shown in FIG. 3 may be the increased force necessary to release the out-dosing in the injection device, i.e. when the injection device release button is reached both the add-on module spring as well as the injection device spring force must be exceeded by the user. In FIG. 3 the spring is designed to elongate the travel of the add-on device's push button relative to the original injection device's push button travel giving a travel of a factor between 1.5 and 5 times the travel of the injection device push-button travel, or by a factor between 5 and 15 or more. For example, if the user is able to depress the actuation push button with e.g. a velocity of 200 mm/sec then the time for depressing the push button e.g. 1.6 mm will be 1.6/200 sec=8 msec. If this time is required to be longer, e.g. 20 msec, the add-on device travel should be at least (20/8)*1.6 mm=4 mm.

FIG. 4 shows a system with a drug delivery device 1 as in FIG. 1 and an add-on data capture module 4, the system being provided with two springs where the one (the serial spring 45) is working in series between the add-on module's actuation push button 43 and the injection device's release button 13 and the other (the parallel spring 44) is working only on the add-on device's actuation button. As appears, this is a combination of the systems shown in FIGS. 2 and 3. Hereby it is possible to make a compromise between injection device tolerance impact on the add-on module reliability and added force to trigger the out-dosing, and still keeping the added elongation of the push button travel and thereby getting the time to monitor the travel of the add-on module's actuation push button to power-up or initialize the electronics or achieve reliable data measurements of the monitoring system.

Alternatively, a biasing force providing a time delay could be provided by a damper mechanism in the add-on module connected to the add-on push button. Hereby the add-on module push button velocity can be slowed down and more time will be given before the movement reaches out-dosing position. The damper mechanism could be of e.g. the hydraulic damper type, pneumatic damper type, friction type, centrifugal type, eddy current type etc. and can be linear or non-linear including valves or not. For the elongation of the travel spring system described above it could be combined with mechanical gear mechanism.

Movement of the add-on module's push button could be detected electronically and if moved too fast for the add-on module electronics to start-up properly the data stored could instead of a measurement of the possible out-dosed amount be information telling the user that an unusual event have happened at that time, possibly a too fast operation of the actuation push button. This could be achieved by e.g. having one or more switches and measuring the movement time for the add-on module push-button travel between these.

The data capture means provided by the system may be of any suitable design as long as it is adapted to work with the specific pen design. For example, the pen may be a non-modified pen or it may be modified to allow for improved data capture (e.g. safer, faster or cheaper). Such modifications may be invisible to the user.

In case the pen is of a non-modified type data capture may be based on detection of indicia in the form of numerical values provided on a rotating dose drum which thus can be read or captured by both a user and the capture means.

A modified pen device may comprise additional dose indicia adapted to be read solely by the capture means. For example, the indicia may be co-arranged on the dose drum, e.g. markings visible also to the user (but not readable) or markings visible only to the capture system.

Alternatively, indicia may be provided on an internal component of the expelling mechanism, the indicia being accessible via a window or opening in the housing.

For example, the capture system may comprise optical detector/decoder means designed to be able to read a code pattern (indicia) or structure on a moving part. As described above, the position of the moving part is related to the amount of drug being out-dosed from the device, either by being an integrated part of the out-dosing mechanism in the injection device itself, or by having a physical connection with a moving part in the out-dosing mechanism in the injection device.

The optical detector shall be arranged in a position relative to the moving part with the code, where its field of view contains a part of the code. When the out-dosing mechanism in the injection device moves this will result in a related movement of the code pattern (detectable by the optical detector/decoder). Hence the code pattern that is in the field of view of the optical detector changes as a consequence of the out-dosing. Since the code pattern is designated to be interpreted by the optical detector/decoder the changes of the code pattern in the field of view of the optical detector can be used to derive the position of the moving part when out-dosing starts and when out-dosing stops (and possibly during the out-dosing). Hence the out-dosing amount can be calculated and subsequently stored and/or communicated by the data acquisition system.

The movement of the moving part with the code pattern may be rotational (around one or more axes), translational (in one or more directions) or a combination hereof.

The code pattern may be either a discrete code (e.g. a binary code), where the code pattern changes instantly for each increment, or it may be an analogue (non-discrete) continuously changing code pattern (e.g. a grey scale code) or a colour code, that changes floatingly from one tone/colour to another tone/colour). The following description is based on a discrete binary code, where the value (0 or 1) is detected based on the intensity of light that reaches the optical detector/decoder. By using more than 1 optical detector or by separating the field of view of a single optical detector a code value with more than 1 bit can be applied. If the code is a 1 bit code, the optical detector must read the code value on the moving part with a time between readings that is fast enough to safely detect every time the code value changes (each out-dosing increment) during the movement of the moving part. If the code has more bits the time between readings can be prolonged and/or a system that is more robust towards tolerances etc. may be made. If the code has e.g. 7 bits a total of 128 different code values are possible. For an injection device with possible out-dosing amounts during one out-dosing between e.g. 1 IU insulin and 80 IU insulin a 7 bits code enables absolute coding/encoding of each increment and the frequency of reading the code may therefore be reduced to be performed only when out-dosing starts and when out-dosing stops.

The code pattern may be integrated on a moving part in multiple different ways. Some examples of methods of producing detectable code patterns directly on the moving part that can be read by the optical detector/decoder are: Printing on the moving part with different colours or tones of grey scale; printing or laser marking of areas on the moving part with dark tone and/or other areas with bright tone; making areas on the moving part that reflects light towards the detector (e.g. by making surface concave or parallel with detector field of view) and/or other areas that reflects light away from the detector (e.g. by making surface convex or non-parallel with detector field of view or by making a rough surface texture); making holes, cut-outs or transparent areas at special fields in the moving part that allows a direct passage of light through the holes/cut-outs/transparent areas to the optical detector and no passage or reduced passage of light outside the holes/cut-outs; making areas on the moving part with special optical properties (e.g. by adding an optical filter, by polarisation or by producing areas with different absorption of light or by producing areas that ensure diffuse reflection of light); making the movable part so it consists or 2 (or more) components, the 2 (or more) components having different optical characteristics (e.g. different colours, brightness, reflection direction, filters, polarisation and/or absorption of light); making holes, cut-outs or transparent areas at special fields in the moving part that allows a direct view of the surface of another component and no passage or reduced view of the surface of the other component outside the holes/cut-outs/transparent areas (the other component may be fixed or moving, the surface of the other component having different optical characteristics than the moving part (e.g. different colours, brightness, reflection direction, filters, polarisation and/or absorption of light); a combination of 2 or more of the embodiments mentioned above.

The code pattern may be placed on a continuous surface of the moving part, or it may be segmented onto multiple broken surfaces of the moving part. If the code pattern is placed on multiple broken surfaces of the moving part, additional optical detectors/decoders are needed.

The code pattern may be designed in multiple different ways. Examples of code patterns, that can be read by the optical detector/decoder are: Binary codes, i.e. codes with radix 2 (e.g. designed as a code based on the binary number system, or as a binary-reflected Gray-code), a binary code with more than 1 bit may be produced by multiple code tracks, or by a single code track with multiple optical detectors/decoders (e.g. by the principle of quadrature encoding), or by a combination hereof; codes with other radices (e.g. 10 which may be based on 10 different colours); analogue (non-discrete) codes (e.g. designed as a grey scale code or as a colour tone code).

Between the optical detector/decoder and the moving part with the code pattern there may be an unobstructed passage of light or the passage of light may be limited by other structures e.g. a frame, a housing or a label material (this is especially relevant in system designs where the moving part is integrated in the injection device itself and the optical detector/decoder is integrated in an external add-on device). In such cases the structure between the moving part with the code pattern and the optical detector/decoder can be removed by having a hole or window in the structure, or the material characteristics for the structure (e.g. a housing or a label) between the moving part with the code pattern and the optical detector/decoder may be selected so it is sufficiently transparent for light to enable detection by the optical detector/decoder. The transparency may be limited to only relevant wave lengths, possibly being non-visual for the normal human eye. Alternatively the add-on device may contain a system that penetrates the structure (e.g. a housing or a label) between the moving part with the code pattern and the optical detector/decoder before the time of detection (e.g. during mounting of the add-on device on or in the injection device).

If the optical detector/decoder is integrated in an add-on device to be mounted on or in the injection device, the moving part with the code pattern may either be an integrated part of the injection device, or it may be an integrated part of the add-on device. If the coded moving part is an integrated part of the add-on device, the coded moving part must be connected to a moving part of the out-dosing mechanism in the injection device, e.g. by use of a mechanical interface, e.g. gear wheels and/or belt drives.

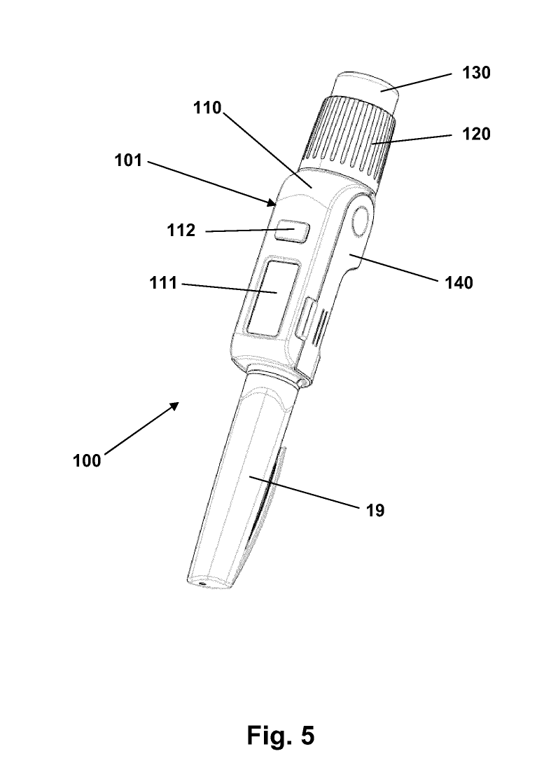

FIG. 5 shows an embodiment of a drug delivery system 100 comprising a drug delivery unit and a data capture unit releasably attachable to each other. The drug delivery device is of the same type as described with respect to FIG. 1 and thus comprises a rotatable dose setting member and a proximally arranged release button (both covered by the data capture unit in FIG. 5). The distal reservoir part of the drug delivery device is covered by a cap member 19. The data capture unit 101 comprises a main portion 110 with a secondary rotatable dose setting member 120 and a secondary proximally arranged release button 130 axially displaceable between an initial, intermediate and actuated position, as well as a lock/release lever member 140 pivotably attached to the main portion. As described with respect to FIG. 3 a spring provides a proximally directed biasing force to the secondary release button.

The main portion comprises electronic vision detection means for capturing numerical dose setting numbers visible in the drug delivery units dose size window (see below), and switch means for initiating data capture, the switch means being actuated when the secondary release button is moved from its initial to its intermediate position. The main portion also comprises an electronic display 111 adapted to show e.g. time and dose size for the last expelling action, as well as a key 112 allowing a user to e.g. toggle between a number of recent time-dose logs. The main portion may further be provided with an output port for wired or wireless upload of data to an external device, e.g. to the users smartphone or a doctors PC.

When the lever is in its open position (see FIG. 6) the data capture unit can be attached to the proximal portion of the drug delivery device, and when the lever is closed (as shown in FIG. 5) and locked to the main portion by releasable mating coupling means 115, 145, the data capture unit is firmly attached to the proximal portion of the drug delivery unit. To ensure that the two units are correctly positioned relative to each other, coupling means (not seen) between the two units ensure that the units can only be locked to each other in a single correct position axially as well as rotational.

When the two units are properly attached to each other the secondary rotatable dose setting member engages the primary dose setting member allowing the user to select a dose in the same way as was no capture unit attached, the dose setting window being visible to the user. Correspondingly, when the dose has been set the user depresses the secondary release button which first activates the switch when moved to its intermediate position, and which subsequently engages the primary release button and releases the expelling mechanism when pushed to its actuated position.

FIG. 6 shows the data capture unit mounted on the drug delivery unit housing portion 10 (with the distal reservoir portion 18 visible) but before the lever 140 has been locked in place. Corresponding to the dose window the data capture unit comprises a cut-out portion 113 with two opposed openings behind which camera and light means are arranged, this providing that both the user and the camera can “see” the number shown in the dose window. To provide a filter (see below) and prevent that the user places e.g. a finger in front of the camera a transparent cover 114 is arranged over the cut-out. The cartridge holder 17 may be permanently fixed to the housing 10 as in a pre-filled device, or it may be releasable mounted allowing a user to replace a drug reservoir (cartridge).

FIG. 7 shows a schematic representation of data capture means adapted to be used in the embodiment of FIGS. 5 and 6. More specifically, a system 200 is shown comprising a dose drum 201 on which numerical indicia are printed, the displayed numerical value representing an actually set dose being visible to a human eye 202 as well as a camera vision system 210 comprising a light source and a digital camera in front of which a lens and filter are arranged. The vision system further comprises processor means (not shown) for analyzing the captured images. As appears, the eye as well as the vision system looks at the same numerical value shown on the dose drum.

The camera system monitors (captures) the display of the mechanical dose drum before and after out-dosing to ensure that only the expelled dose is recorded. The images are analyzed and dose values interpret. If the difference is >0 an out-dosing has taken place and the out-dosed amount is registered and stored. Together with the out-dosed amount the time when the images are captured (each or one or a derived expression of one or both) are registered. The out-dosed amount and time when out-dosing took place is associated to each other, i.e. put into the same record or linked to each other and stored. The recorded values can itself or after data processing be displayed on the pen and/or compared with derivative of planned injection signature used for assuring the patient is in compliance with prescribed treatment either in real time (e.g. by reminder alarm) and/or later retrospectively (e.g. by the patient or a care taker person be read out or communicated to another device

The shown camera system comprises at least one image sensor 211 with optics 212 and at least one light source 213 (e.g. a LED). The camera system (i.e. image sensor system) including the light source utilizes light either inside and/or outside the visible (photometric) range. In an exemplary system the camera system utilizes light outside the visible range. The user observes the dose drum through a first light pathway in which light is let through a first optical filter 214 where the wavelength for the light used for the measuring system is dampened. The image sensor measuring system operates in a second light pathway to/from the dose drum which is illuminated by a light source utilizing light in a narrow range within the above dampened spectral range. This light is reflected from the dose drum and led through a second optical filter 215 which is dampening wavelengths that could disturb the image measuring system. The light from the dose drum reaching the camera (i.e. image sensor) may be conditioned by an optical system (e.g. a lens) to fit with a certain area and focal length and distortion on the camera image sensor. The second pathway constitutes an angle to the first pathway between 0 and 90 degrees and preferable greater than 40 degrees and less than 75 degrees.

Alternatively a camera system may be used capturing images of a dose drum using two cameras placed opposite to each other with two light sources also opposite to each other.

The two-camera systems (including light sources) are image capture operated either time multiplexed or simultaneously with each camera & light system operating on different specific wavelengths within the above dampened wavelengths. Depending on the specific geometry of the system, when using one camera system on a scale drum it may not be possible for the camera system to capture part of an image below the horizon. This could be avoided with a two-camera system. The two-camera system and the associated controller system would also have the possibility to compare the two opposite images and identify the common area in order to enhance the safety of the system.

In the above description of the preferred embodiment, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A drug delivery system, comprising:
expelling device for expelling a dose of drug from a reservoir,
an electronically controlled capturing system for capturing data representing a property related to the amount of drug expelled from a reservoir by the expelling means,
switch device for starting initialization of the capturing system,
a release member for releasing or driving the expelling means and adapted to be moved between:
an initial position, and
an actuated position in which the expelling means is released or driven,
an actuation member adapted to be moved by a user between:
an initial position,
an intermediate position in which the switch means is actuated to start initialization of the capturing system, and
an actuated position in which the release member is actuated,
actuation biasing means providing a biasing force against movement of the actuation member between its initial and intermediate position,
whereby the capturing system is allowed to initialize during the actuation member's movement between the intermediate position and the actuated position.

2. A drug delivery system as in claim 1, wherein the expelling device can be set to deliver a user selected dose size, the selected dose size being represented by indicia which can be captured by a detection device.

3. A drug delivery system as in claim 2, wherein the expelling device comprises a rotatable member on the surface of which indicia are arranged, the indicia representing the set dose size being positioned at a given capture position allowing capture thereof by the detection device.

4. A drug delivery system as in claim 3, wherein the rotatable member rotates during expelling of a set dose, the indicia at a given point in time representing, directly or indirectly, the expelled dose.

5. A drug delivery system as in claim 3, wherein the indicia is a numerical value which can be read or captured by both a user and the detection device.

6. A drug delivery system as in claim 3, wherein two indicia are provided on one or two rotatable members, one indicia being readable by a user, the other being readable by the detection device.

7. A drug delivery system as in claim 1, wherein the actuation member during movement between its intermediate and actuated position directly or indirectly is brought in contact with the release member.

8. A drug delivery system as in any of claim 1, wherein the biasing device comprises a spring acting on the actuation member.

9. A drug delivery system as in claim 8, wherein the spring has a stationary portion being supported by a component of the system which does not move during setting and expelling of a dose of drug.

10. A drug delivery system as in claim 1, further comprising:
release biasing device providing a biasing force against movement of the release member between its initial and actuated position,
wherein the biasing device are configured to provide a step-formed increase in the biasing force acting on the actuation member during movement between its intermediate and actuated position.

11. A drug delivery system as in claim 1, comprising a housing in which the expelling device, the release member, the actuation member, the electronically controlled capturing system, the switch device, and the actuation biasing device are arranged.

12. A drug delivery system as in claim 1, comprising a drug delivery unit and a data capture unit releasably attachable to each other, the drug delivery unit comprising:
the expelling device, and
the release member,
the data capture unit comprising:
the actuation member (130),
the electronically controlled capturing system,
the switch device, and
the actuation biasing device.

13. A drug delivery system as in claim 1, comprising a drug reservoir.

14. A drug delivery system as in claim 1, the system being configured to receive a replaceable drug reservoir in the form of a drug cartridge.
